(19) United States
(12) Patent Application Publication
(10) Pub. No.: US 2014/0357945 A1
(43) Pub. Date: Dec. 4, 2014

(54) Laparoscopic Trocar with Ventriculoperitoneal Shunt Entry Port

(71) Applicant: Edward Duckworth, Houston, TX (US)

(72) Inventor: Edward Duckworth, Houston, TX (US)

(21) Appl. No.: 14/292,506

(22) Filed: May 30, 2014

Related U.S. Application Data
(60) Provisional application No. 61/828,686, filed on May 30, 2013.

(51) Int. Cl. A61M 27/00; A61B 17/34; A61B 1/313

(52) U.S. Cl. CPC A61M 27/006; A61B 1/313; A61B 17/3415
USPC 600/104

(57) ABSTRACT

An example laparoscopic trocar assembly can be used for insertion of a ventriculoperitoneal (“VP”) shunt into an abdomen and comprises an optical system a trocar sleeve to allow a surgeon to see abdominal tissue being punctured and see into an abdominal cavity. An example trocar assembly comprises an entry port in the trocar's side and a stylet to insert a VP shunt tube into an abdomen. An example trocar assembly comprises a transparent dissecting obturator with a sharp edge to puncture an abdominal wall and an optical system or camera to allow a surgeon to see inside of an abdominal cavity to avoid injury to blood vessels and internal organs during surgery. An example trocar comprises a side channel through which a VP shunt tube can be inserted into an abdominal cavity at the same point of insertion as the trocar.

This application claims the benefit of provisional application 61/828,686.

### BACKGROUND

Hydrocephalus is a condition that occurs when there is excess cerebrospinal fluid (CSF) in the ventricles of the brain. Normally, excess CSF drains away from the brain and absorbed by the body. For people with hydrocephalus, this doesn't happen, and the fluid builds up in the ventricles. The treatment for hydrocephalus is to perform a surgery to release the pressure on the brain caused by the excess CSF. If the excess fluid is not caused by a tumor, the normal treatment for relieving the pressure on the brain requires draining the fluid through a shunt operation. A hole is drilled in to the skull of the patient and a VP shunt is inserted. The tube is then is placed underneath the skin and inserted into the abdominal cavity. The excess fluid is drained into the abdomen.

A “Ventriculoperitoneal (VP) shunt” is a device used to relieve pressure from the brain caused by fluid accumulation. It comprises a catheter that passes into the brain and is attached to a one way valve on the other end. A shunt tube attached to the other end of the valve runs underneath the skin down to the abdomen.

A “stylet” is a thin metal probe for inserting into or passing through a needle, tube, or catheter for inserting into a soft, flexible catheter to make it stiff as the catheter is placed in a vein or passed through an orifice of the body.

An “obturator” is a stylus or removable plug used during the insertion of many tabular instruments.

In the United States, tens of thousands of shunt operations are performed annually to treat hydrocephalus in children and adults. The most common type of shunt operation is a ventriculoperitoneal shunt, in which a tube is inserted into the cavities of the brain, attached to a valve, and tunneled under the skin to the abdomen, where the tube is then inserted into the “belly” (peritoneal cavity). Neurosurgeons typically perform this operation without the assistance of a general surgeon.

Most commonly, a neurosurgeon will employ a “mini-laparotomy” technique in which a 6-8 cm incision in the abdominal wall is made, and “open” dissection carried through the rectus muscle and fascia to enter the abdomen for shunt insertion. A typical shunt tube is approximately 2-3 mm in diameter. Despite this being a seemingly safe (albeit large and cosmetically undesirable) strategy, complications do occur, including malpositioning of the shunt, (most commonly in the pre-peritoneal space), bowel injury, and hernias.

Modern general surgery often calls for minimally invasive laparoscopic surgery so as to limit trauma to the patient, as well as to minimize the amount of time necessary to recover. A common instrument in laparoscopic surgery is a trocar, a pointed device used to create a perforation in tissues so that the surgeon can access the interior of the body. Trocars serve several different functions. They allow interior access and open the tissue around the perforation so that other surgical devices can enter into the body cavity.

A minority of neurosurgeons will use a “split abdominal trocar” to enter the belly. It is made with a solid plastic pointed cylindrical stylet, and a split (incomplete) metal outer sleeve which allows for removal of the trocar after the shunt tubing is inserted through it. The trocar is effective when in experienced hands, bat is a blind procedure, requires a steep learning curve, and has not enjoyed widespread adoption, because of fears of complications such as bowel perforation.

Over the past decade, laparoscope has become widely accepted in general surgery, if not the standard of care for many intraabdominal procedures. The techniques and technology have advanced to allow for fewer and smaller incicisions, as well as fester and safer surgeries. One significant advance has been the way in which the peritoneal cavity is first entered. In the past, open dissection followed by insertion of a blunt trocar through the navel would be used to provide the initial entry and insufflation of gas (allowing safe entry of all the other ports—under direct visualization, and with the cavity expanded by gas). Another technique employed the use of a “Varies needle” to percutaneously enter the peritoneal cavity (blindly) and insufflate CO2. More recently, devices have emerged that allow for trocar dissection through the abdominal wall under direct visualization with the laproscope, by employing a clear trocar within a port housing. Even in the setting of a previously operated abdomen (with potentially dangerous intrabdominal scarring/adhesions), the dissection is done under direct visualization and as slowly or quickly as desired. Entering the abdomen through a small (5-10 mm) incision is safer with visualization.

The devices described above require either blind insertion or do not allow for release of tubing once it is inserted.

### BRIEF DESCRIPTION OF THE DRAWINGS

FIG. 1 shows an embodiment comprising a laparoscopic trocar with a split side channel and cross sectional views show an optical system passing through the center of the trocar and a VP shunt passing through the split side channel.

FIG. 2 shows a trocar with a VP shunt tube entering the side channel through the stylet for insertion into the abdominal cavity.

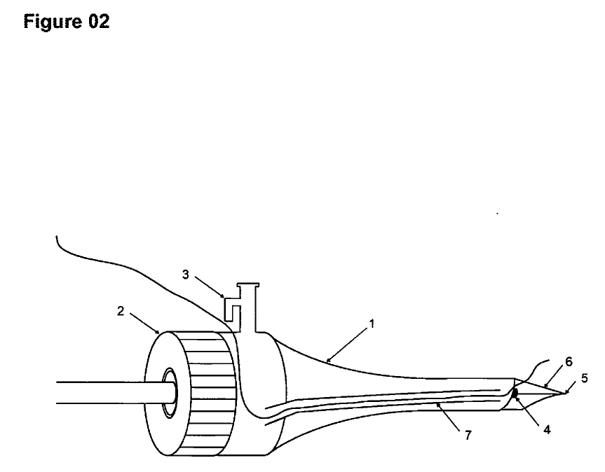

FIG. 3 shows a VP shunt's position on a human and the shunt's entry into the abdomen via a side channel in a trocar.

FIG. 4 shows an embodiment comprising a laparoscopic trocar with a split side channel.

### DETAILED DESCRIPTION

A laparoscopic trocar for VP shunt surgery and insertion of the VP shunt into the abdominal cavity is described in detail with reference to the attached drawings.

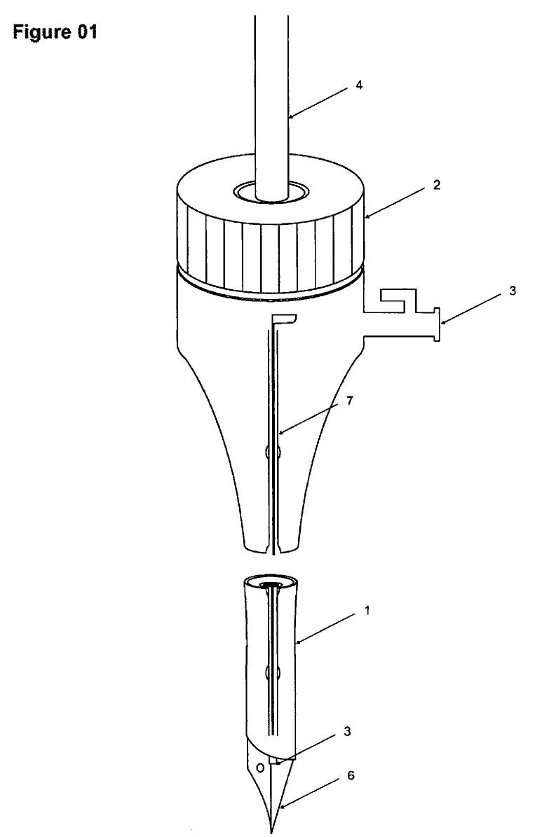

FIG. 1 shows orthographic and cross sectional views of an embodiment comprising an outer trocar sleeve 1 and a laparoscopic trocar 2.

The trocar sleeve 1 comprises a cylindrical hollow tube made from biologically compatible inflexible material such as stainless steel or a suitable transparent material. The trocar sleeve 1 comprises a split side channel 7 providing an entry path for a stylet 20 and a passage way for inserting a VP shunt tube into the abdomen. The side channel 7 runs along the side of the cylindrical trocar sleeve 1 and enters the abdominal cavity at the point of piercing. The side channel 7 has a diameter sufficient to insert a VP shunt tube which has been run from the patient's brain ventricle, under the skin to the abdominal cavity to drain the excess CSF fluid. This provides access of the VP shunt into the abdominal cavity at the point of visual piercing. A trocar sleeve 1 can comprise an infusion port 3 mounted on its side through which carbon dioxide gas can be delivered into the abdominal cavity.

The trocar 2 comprises a cylindrical hollow tube with a sharp dissecting end 6 that is sufficiently sharp to be surgically inserted through layers of human flesh. The cylindrical hollow trocar 2 has a central opening of a sufficient diameter to encompass an optical system 4 such as a telescope or a camera and a light projecting element. The sharp front end 6 is made of transparent material such as glass or plastic and is shaped to form a light transmitting and imaging element for projecting light into the abdominal cavity and for transmitting images to the optical system. The entire trocar 2 can be made from the same transparent material. In one embodiment an optical system 4 comprises a light receptor comprising a plurality of light receiving input ends and a light source comprising a bundle of fiber optic light coupling elements. In another embodiment an optic system 4 comprises a miniaturized medical light telescope for surgical use. A camera provides images to a video monitor (not shown) that a surgeon can see while the trocar 2 is penetrating skin and proceeding into the abdominal cavity.

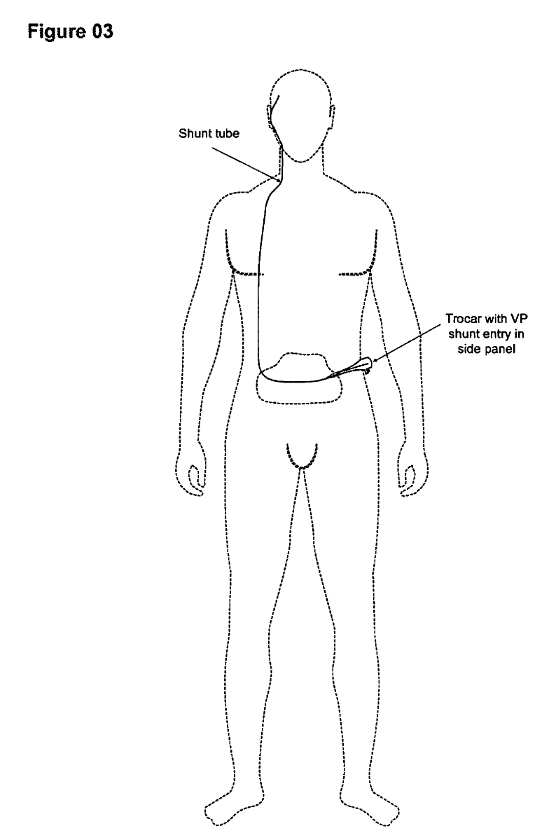

FIG. 3 illustrates the use of a VP shunt tube in ventriculoperitoneal shunting surgery to drain excess fluid from the brain to the abdomen.

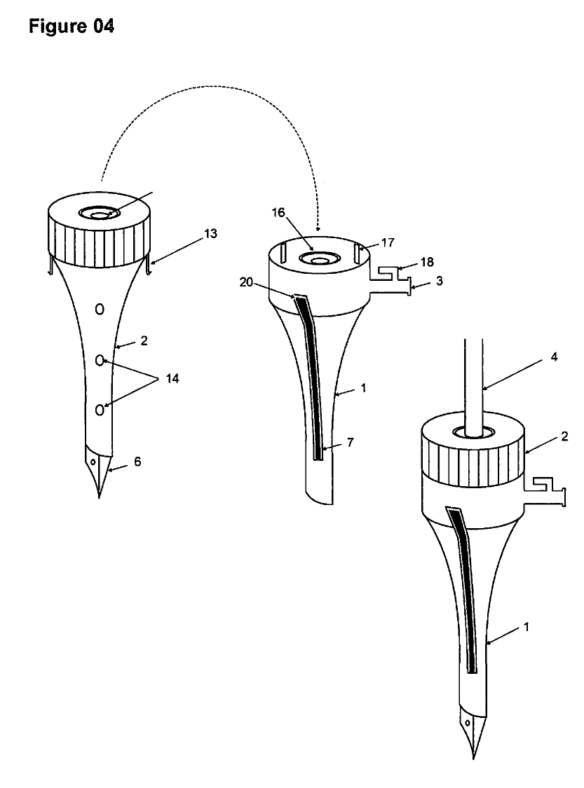

FIG. 4 shows an embodiment comprising an outer trocar sleeve 1 and a laparoscopic trocar 2 shown as separate components and also shows the two components mated together with the trocar 2 inserted into the trocar sleeve 1.

The trocar sleeve 1 comprises a cylindrical hollow tube made from biologically compatible inflexible material such as stainless steel or a suitable transparent material. The trocar sleeve 1 comprises a split side channel 7 providing a passage way for a stylet 20 and an entry path for inserting a VP shunt tube into the abdomen. The side channel 7 runs along the side of the cylindrical trocar sleeve 1 and enters the abdominal cavity at the point of piercing. The side channel 7 has a diameter sufficient to insert a VP shunt tube which has been run from the patient's brain ventricle, under the skin to the abdominal cavity to drain the excess CSF fluid. This provides access of the VP shunt into the abdominal cavity at the point of visual piercing. A trocar sleeve 1 can comprise an insufflation port 3 mounted on its side through which a fluid, such as carbon, dioxide gas, can enter the trocar sleeve's central cavity and pass through the trocar's insufflation holes 14 into the trocar's central opening and flow into the abdominal cavity. The insufflation port 3 comprises an insufflation port valve 18 that regulates fluid flow into the trocar sleeve 1. Trocar sleeve 1 comprises a trocar port 16 into which trocar 2 fits. Trocar sleeve 1 comprises snap attachment ports 17 into which a trocar's snap attachments 13 fit, locking the trocar 2 into place inside the trocar sleeve 1.

The trocar 2 comprises a cylindrical hollow tube with a sharp dissecting end 6 that is sufficiently sharp to be surgically inserted through layers of human flesh. The cylindrical hollow trocar 2 comprises a central opening having at its back end a camera light source port 11 with a rubber valve through which an optical system 4 can enter. The sharp front end 6 is made of transparent material such as glass or plastic and is shaped to form a light transmitting and imaging element for projecting light into the abdominal cavity and for transmitting images to the optical system. The entire trocar 2 can be made from the same transparent material. In one embodiment, an optical system 4 comprises a light receptor comprising a plurality of light receiving input ends and a light source comprising a bundle of fiber optic light coupling elements. In another embodiment an optic system 4 comprises a miniaturized medical light telescope for surgical use. A camera provides images to a video monitor (not shown) that a surgeon can see while the trocar 2 is penetrating skin and proceeding into the abdominal cavity. The trocar 2 comprises a snap attachment 13 for docking trocar 2 to trocar sleeve 1. The trocar 2 comprises insufflation holes 14 in fluid communication with the central opening tor introducing a fluid, such as carbon dioxide gas, in to the abdominal cavity.

An Optical Entry Shunt Insertion Port according to an embodiment can improve the safety and efficiency of the abdominal portion of the VP shunt procedure. An embodiment incorporates the functionality of a split trocar (allowing a tube to be inserted through it, but without being “trapped” in the device), with the assurance of directly visualizing each layer of the abdominal wall as it Is traversed.

An embodiment comprises a trocar device for laparoscopic placement of shunt tubing wherein a laparoscope is integrated, and allows a shunt tube to be inserted into the abdomen after the trocar enters the abdomen. An embodiment allows a user to visually identify the tissue through which the trocar is being dissected and also allows a user to insert shunt tubing into the abdomen through the same minimally invasive incision. An embodiment comprises a camera to visualize the insertion of the trocar through layers of tissue as well as the insertion of the shunt in one procedure, avoiding the exchange of instruments during the procedure. The optical entry allows for full visualization of the procedure at all times, avoiding potential unintentional perforation of abdominal, organs and tissues. The combination of all functions into one device enables a faster and safer procedure, avoiding complications.

What is claimed is:

1. A trocar for penetrating a patient's abdomen, comprising:
a tubular outer trocar sleeve 1 having a front end, a back end, and a hollow interior; and
a tubular laparoscopic trocar 2 having a front end, a back end, an exterior surface, and a hollow interior;
wherein said trocar sleeve and said laparoscopic trocar are shaped to be mated together with said laparoscopic trocar 2 inserted into said trocar sleeve's hollow interior 1 from said trocar sleeve's back end;
wherein said trocar sleeve 1 comprises biologically compatible inflexible material;
wherein said trocar sleeve 1 comprises a split side channel 7 extending along said trocar sleeve's exterior surface approximately from said trocar sleeve's back end approximately to said trocar sleeve's front end;
wherein said side split channel provides a passageway for a stylet and provides an abdominal cavity entry path for inserting a VP shunt tube;
wherein said trocar's front end comprises a sharp dissecting end made of transparent material such as glass or plastic and wherein said sharp dissecting end is shaped to form a light transmitting and imaging element; and
wherein at said trocar's back end, said trocar's hollow interior comprises a camera light source port through which an optical system 4 can enter.
